(19) United States
(12) Patent Application Publication
(10) Pub. No.: US 2023/0184758 A1
(43) Pub. Date: Jun. 15, 2023

(54) AUTOIMMUNE HEPATITIS BIOMARKERS

(71) Applicant: ACADEMISCH ZIEKENHUIS LEIDEN (H.O.D.N. LUMC), Leiden (NL)

(72) Inventors: Tamas PONGRACZ, Leiden (NL); Maaike BIEWENGA, Oegstgeest (NL); Manfred WUHRER, Voorschoten (NL); Noortje DE HAAN, Copenhagen (DK); Bart VAN HOEK, Voorschoten (NL)

(21) Appl. No.: 18/080,342

(22) Filed: Dec. 13, 2022

(30) Foreign Application Priority Data
Dec. 14, 2021 (NL) 2030135

(51) Int. Cl. G01N 33/564; G01N 33/576

(52) U.S. Cl. CPC G01N 33/564; G01N 33/5767; G01N 2800/50; G01N 2440/38

(57) ABSTRACT

The present invention relates to methods for diagnosing and/or predicting the risk of developing autoimmune hepatitis (AIH) in a subject. The methods are based on the inventors' identification of a novel biomarker for AIH. The invention further comprises the use of said biomarker in the methods disclosed herein, and kits comprising agents for detecting said biomarker. The invention also relates to a method of treating AIH in a subject, wherein the subject has been diagnosed with AIH or determined to be at risk of developing AIH using the methods disclosed herein.

### CROSS-REFERENCE TO RELATED APPLICATIONS

This application claims priority under 35 U.S.C. § 119 to Netherlands Application No. 2030135 filed Dec. 14, 2021, which is incorporated herein by reference in its entirety.

### FIELD

The present invention relates to methods for diagnosing and/or predicting the risk of developing autoimmune hepatitis (AIH) in a subject. The methods are based on the inventors' identification of a novel biomarker for AIH, namely A4GS. The invention further comprises the use of said biomarker in the methods disclosed herein, and kits comprising agents for detecting said biomarker.

### BACKGROUND

AIH is a chronic autoimmune disease of the liver, characterised by autoantibodies and elevated total IgG¹. Diagnosis of AIH is based on a scoring system which combines, amongst others, autoantibodies, IgG level and the results of a liver biopsy^{2,3}. However, it is often still difficult to distinguish AIH from other (autoimmune) liver diseases such as primary biliary cholangitis (PBC), primary sclerosing cholangitis (PSC) or non-alcoholic fatty liver disease (NAFLD), of which the prevalence is increasing. Additionally, a liver biopsy is an invasive procedure with risk of bleeding.

The aim of treatment of AIH is biochemical and histological remission, as this is associated with a reduced disease progression and better long-term survival^{4,5}. While a liver biopsy is informative in disease monitoring, in clinical practice disease activity is determined by aspartate transaminase (AST), alanine transaminase (ALT) and IgG level due to the high risks related to repeated liver biopsies. In some patients only partial biochemical remission can be obtained and relapses can occur especially after treatment is stopped⁶.

Accordingly, a new, non-invasive AIH diagnostic method would be highly desirable as it may reduce the need for liver biopsy and provide a more tailored and less invasive method of monitoring of the patients. The present invention aims to provide such a method.

### SUMMARY

The post-translational modification of proteins by N-glycosylation adds a layer of functional complexity to the majority of human proteins. Differences in N-glycan structures may influence the plasma half-life of glycoproteins as well as their functions. As a major part of the plasma glycoproteins are synthesized—and glycosylated—in the liver, liver disease-induced glycosylation changes of plasma glycoproteins are of high interest. Previously, specific glycosylation patterns have been associated with liver cirrhosis and risk of hepatocellular carcinoma, which form the basis of the clinically used GlycoCirrhoTest^{7,8}. Distinct changes in plasma protein glycosylation have also been associated with several (autoimmune) diseases including rheumatoid arthritis (RA), inflammatory bowel disease (IBD) and type II diabetes⁹⁻¹².

IgG is a non-liver-derived plasma glycoprotein of which elevated levels are found in AIH⁴. The various effector functions of IgG are highly dependent on the glycosylation of the Fc region in its constant domain. For example, fucosylation of the IgG Fc glycan limits IgG mediated antibody dependent cellular cytotoxicity by lowering the affinity of IgG for the FcγRIII receptor¹³. Furthermore, the degree of Fc glycan galactosylation is strongly associated with inflammation. This protein-specific glycan trait decreases with aging as well as in several (autoimmune) diseases, and dynamically relates to disease behaviour as exemplified in RA and IBD¹⁴⁻¹⁹.

As shown in the Examples section of the present disclosure, the inventors have extensively studied global plasma protein N-glycosylation (the total plasma N-glycome, (TPNG)) as well as IgG-specific glycosylation. Surprisingly, the inventors have been able to identify a single glycosylation marker that is unique for patients with AIH. The inventors compared the plasma and IgG glycan levels of patients with AIH to healthy controls, as well as to patients with PBC, PSC, NAFLD and viral or alcoholic hepatitis with different stages of cirrhosis, i.e., without cirrhosis, compensated cirrhosis or decompensated cirrhosis (WC, CC and DC, respectively). When comparing the levels, the inventors took into account known confounders of glycosylation, including sex, age, cirrhosis, and the use of immunosuppressive medication. These comparative studies allowed the inventors to identify for the first time a single glycosylation profile with differential diagnostic potential for AIH. Specifically, the inventors have identified that patients with AIH have higher levels of sialylation per galactose on tetraantennary glycans. In other words, the patients with AIH were found to have higher levels of A4GS. Advantageously, the inventors have found that the levels of A4GS are neither confounded by the age and sex of the subject, making A4GS a biomarker that may be easily applied in diagnostics.

Accordingly, in the first aspect, the present invention provides a method for diagnosing autoimmune hepatis (AIH) in a subject, or predicting the risk of AIH developing in a subject, the method comprising:

a) determining the level of A4GS in a sample from the subject; and

b) comparing the level of A4GS in the sample to a reference level, wherein an increase in the level of A4GS in the sample as compared to the reference level is indicative of the subject having AIH, or being at risk of developing AIH.

Suitably, the sample may be a biological fluid sample.

Suitably, the sample may be selected from the group consisting of blood, urine and saliva, optionally wherein the blood sample may be plasma or whole blood.

Suitably, the plasma sample may have been subjected to N-glycan release from plasma proteins and linkage-specific chemical sialic acid derivatization.

Suitably, the reference level may be the level of A4GS in a control sample. The control sample may be from one or more control subject. The control subject may be an individual that does not have AIH and/or is not at risk of developing AIH.

Suitably, the subject may be a mammal (for example human, monkey, rat, mouse, mink, rabbit, guinea pig, pig, dog, cat, goat, sheep, horse or cow).

Suitably, the subject may not have, or may be believed to not have, viral or alcoholic hepatitis.

Suitably, the subject may be symptomatic or asymptomatic.

Suitably, the symptomatic subject may have a symptom selected from the group consisting of fatigue, jaundice, abdominal pain, joint pain and/or swelling, mild flu-like symptoms, itching, large abdomen due to enlarged liver and/or spleen, and spiderlike blood vessels in the skin.

Suitably, the level A4GS may be determined by mass spectrometry, high-performance liquid chromatography, capillary (gel) electrophoresis with laser induced fluoresces detection, hydrophilic interaction liquid chromatography (for example with fluorescence or UV detection), or an ELISA based assay (for example an ELISA based assay using glycan binding proteins such as lectins or antibodies).

Suitably, the method may further comprise determining the level of aspartate transaminase (AST), alanine transaminase (ALT) and/or IgG in the sample. Thus, suitably, the level of AST, ALT and/or IgG may be used as an additional biomarker for diagnosing or predicting the risk of the AIH developing in a subject.

In another aspect, the invention provides a method for diagnosing AIH in a subject or predicting the risk of the AIH developing in a subject, and treating AIH in a subject, the method comprising the steps of:

a) determining the level of A4GS in a sample from the subject; and

b) comparing the level of A4GS in the sample to a reference level;

c) identifying the subject as having AIH if the levels of A4GS in the sample are increased compared to the reference level; and

d) administering to the subject that has been identified as having AIH or being at risk of having AIH a treatment for AIH.

Suitably, the treatment may be a prophylactic treatment or disease modifying treatment.

Suitably, the treatment may be an immunosuppressant, optionally the immunosuppressant may be prednisone, prednisolone, budesonide, azathioprine, mycophenolate mofetil, 6-thioguanine, 6-mercaptopurine, rituximab and/or tacrolimus.

In another aspect, the invention provides a method of treating AIH in a subject, the method comprising the steps of administering an AIH treatment to a subject, wherein the subject has been diagnosed or determined to be at risk of AIH, or identified as having AIH by a method as described herein.

In another aspect, provided herein is a kit comprising a detectably labelled agent that specifically binds to A4GS.

Suitably, the kit further comprises a detectably labelled agent that specifically binds to aspartate AST, ALT and/or IgG.

In another aspect, provided herein is the use of a A4GS as a biomarker for AIH.

Except for where the context requires otherwise, the considerations set out in this disclosure should be considered to be applicable to the recombinant protein, nucleic acid sequence, expression vector, cell, and/or exosome in accordance with the invention, and the uses thereof.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE DRAWINGS

Embodiments of the invention are further described hereinafter with reference to the accompanying drawings, in which:

FIG. 1 (A) is a schematic representation of common monosaccharide constituents of human N-glycans. (B) Shows a schematic representation of the three general types of N-glycans. (C) A schematic representation of N-glycosylation derived traits, and their abbreviations, representing common biosynthetic pathways.

FIG. 2 shows main associations found between AIH patients and healthy controls. (A) Is a volcano plot based on the 155 derived glycosylation traits. (B-E) Show relative abundance differences of derived glycosylation traits: (B) IgG1 galactosylation, (C) THy, (D) CB (E) A4GS, between AIH and healthy controls and other liver disease groups. AIH is the reference group against which comparisons are shown. P-values, ORs and corresponding 95% CIs for the associations are shown in Table 2. Boxplots represent the median, the interquartile range, whilst whiskers correspond to the first and third quartiles (25^{th} and 75^{th} percentiles) and extend from the hinges to the largest or smallest value no further than 1.5× the interquartile range. *, **, ***, ****: p-value<0.05, 0.01, 0.001, 0.0001, respectively.

FIG. 3 shows associations between cirrhosis severity and bisection in alcoholic and viral hepatitis patients. Relative abundance differences of glycosylation derived traits (A) CB and (B) CirrhosisTest. Statistical tests and boxplots as described in FIG. 2.

FIG. 4 shows correlations between AIH disease activity and glycosylation. Relative abundance differences of derived glycosylation traits: (A) IgG1 galactosylation, (B) THy, (C) CB and (D) A4GS. Statistical tests and boxplots as described in FIG. 2.

FIG. 5 shows longitudinal changes associated with AIH disease activity. Relative abundance differences of glycosylation derived traits: (A) THy and (B) A4GS.

FIG. 6 shows age and sex distribution across disease groups, and in an age and sex matched subset of AIH, CC and DC. (A) Age distribution in the cohort. (B) Age distribution across disease groups, as stratified per sex. (C-H) Age and sex distribution in an age and sex matched subset of AIH, CC and DC, for the visualization of potential confounding effect of age and sex. No significant age and sex differences were observed. ns: no significance. Statistical tests and boxplots as described in FIG. 2.

FIG. 7 shows IgG1 galactosylation for an age and sex matched subset of AIH, CC and DC, for the visualization of potential confounding effect on age and sex, when compared to effects seen on FIG. 2B. A disease effect could be confirmed for the hepatitis patients with decompensated cirrhosis (D, F), unlike for hepatitis patients with compensated cirrhosis, for which a potential age-effect could not be excluded (C, E). Statistical tests and boxplots as described in FIG. 2.

FIG. 8 shows A4GS for the age and sex matched AIH patients and HC, for the visualization of potential confounding effect on age and sex. A disease effect could be confirmed for AIH. Statistical tests and boxplots as described in FIG. 2.

FIG. 9 shows a comparison of N-glycosylation signatures in patients with different degrees of cirrhosis. Relative abundance differences of glycosylation derived traits (A) IgG1 galactosylation, (B) THy, (C) CB and (D) A4GS. HC: healthy controls; AIH/WC: autoimmune hepatitis without cirrhosis; AIH/CC: autoimmune hepatitis with compensated cirrhosis; AIH/DC: autoimmune hepatitis with decompensated cirrhosis. Patients without AIH in WC correspond only to viral hepatitis, whereas CC and DC are a mixture of alcoholic and viral hepatitis (Table 1). Statistical tests and boxplots as described in FIG. 2.

FIG. 10 shows the results of longitudinal studies. No association with AIH disease activity is observed. Relative abundance differences of glycosylation derived traits: (A-B) IgG1 galactosylation, (C) THy, (D-E) CB and (F) A4GS.

FIG. 11 shows principal component analysis (PCA) scores plot based on the distribution of sample plates per standards and disease groups. Displayed is the 1st PC against the 2nd PC (covering 41.8% and 13.2% of the variation, respectively), with the ellipse indicating the 95% confidence interval. The separation of standards is not or hardly driven by systematic batch effects: the standards do not cluster per plate, indicating that the variation between standards is largely random and not batch-associated.

### DETAILED DESCRIPTION

The present description provides a method for diagnosing autoimmune hepatis (AIH) in a subject, or predicting the risk of the AIH developing in a subject, the method comprising:
a) determining the level of A4GS in a sample from the subject; and
b) comparing the level of A4GS in the sample to a reference level, wherein an increase in the level of A4GS in the sample as compared to the reference level is indicative of the subject having AIH, or being at risk of developing AIH.

This method is based on the inventors' finding of a novel biomarker that enables differential diagnosis of AIH. Currently, diagnosing AIH, without performing an invasive liver biopsy is difficult, as other diseases such as primary biliary cholangitis (PBC), primary sclerosing cholangitis (PSC) or non-alcoholic fatty liver disease (NAFLD) have a very similar clinical and biochemical presentation. Surprisingly, however, the inventors have found a single biomarker which is increased in AIH as compared to controls and many other liver diseases that otherwise have a similar clinical presentation.

AIH is a chronic inflammatory liver disease, which has a prevalence of approximately 1/10,000 in the United States and Europe and accounts for 2-6% of all liver transplantations in these countries. It is typically caused by autoantibodies (such as anti-antinuclear antibody (ANA), anti-smooth muscle antibody (SMA), anti-liver kidney microsomal antibodies (LKM-1, LKM-2, LKM-3), anti-soluble liver antigen (SLA), liver-pancreas antigen (LP), and anti-mitochondrial antibody (AMA)), environmental triggers (such a virus, taking certain medications, or coming into contact with other toxins), or associated with a genetic predisposition (i.e. genes which may make it easier for a trigger to set off the disease). In the context of the present disclosure AIH may also be referred to as “the disease” or “the disorder”.

Suitably, in the context of the present disclosure, the subject may not have or may be believed not to have viral or alcoholic hepatitis. Whilst sometimes viral or alcoholic hepatitis (for example viral or alcoholic hepatitis without cirrhosis) may be also associated with increased A4GS, the clinical presentation of viral or alcoholic hepatitis is typically quite different to that of AIH. Therefore the skilled person would not have a difficulty in differentiation between viral or alcoholic hepatitis and AIH. Furthermore, by way of example a subject with viral hepatitis (for example hepatitis A, B, C, D or E) will typically have viral DNA, antigens, and/or antibodies. Thus, in the context of the methods of the present invention, suitably the subject might not have viral DNA, antigens, and/or antibodies. Alternatively or additionally, the subject might not have a history of alcohol abuse. It will be appreciated that in this context, viral DNA or viral antigens, refers to DNA and/or antigens that come from viruses that cause hepatitis, and antibodies refers to antibodies against such antigens.

In the context of the present disclosure, the subject is typically a mammal. The mammal may be for example a human, a monkey, a rat, a mouse, a mink, a rabbit, a guinea pig, a pig, a dog, a cat, a goat, a sheep, a horse or a cow. More suitably, the subject is a human. The terms “subject”, “individual”, and “patient” are used herein interchangeably.

The subject can be symptomatic (e.g., the subject presents symptoms associated with AIH), or the subject can be asymptomatic (e.g., the subject does not present symptoms associated with AIH). Symptoms of AIH include but are not limited to fatigue, jaundice, abdominal pain, joint pain and/or swelling, mild flu-like symptoms, itching, large abdomen due to enlarged liver and/or spleen, and/or spiderlike blood vessels in the skin.

The subject may be diagnosed with, be at risk of developing, or present with symptoms of, AIH. In a subject that has been diagnosed with AIH the method disclosed herein may simply confirm that the subject has AIH. In some embodiments, the subject does not have AIH, but may or may be believed to be at risk of AIH (or higher likelihood of having AIH), for example due to known or suspected contact with an environmental trigger or genetic predisposition, and/or due to having symptoms of AIH. A subject that is at risk of AIH may have been, for example, in contact with an environmental trigger known or believed to be cause or increase the chance of having AIH, or the subject may be known or at risk of having (for example based on family history) a genetic predisposition, such as a mutation known or believed to cause and/or increase the chance of having AIH.

In general, the methods described herein are in vitro methods that are performed using a sample that has already been obtained from the subject (i.e. the sample is provided for the method, and the steps taken to obtain the sample from the subject are not included as part of the method).

Suitably, the sample may be a biological fluid sample, such as a blood, urine or saliva sample. More suitably, the blood sample may be plasma or whole blood. Most suitably, the sample is a plasma sample. Suitably, the plasma sample may have been subjected to N-glycan release from plasma proteins and linkage-specific chemical sialic acid derivatization. It will be appreciated that the sample may be subjected to N-glycan release from plasma proteins and linkage-specific chemical sialic acid derivatization prior to determining the level of A4GS in a sample. Suitably the sample may be prepared as described in Vreeker, G. C. M. et al. 2018 (doi:10.1021/acs.analchem.8b02391), which is incorporated herein by reference.

Methods for obtaining biological fluid samples (e.g. whole blood, serum, plasma, urine etc) from a subject are well known in the art. For example, methods for obtaining blood samples from a subject are well known and include established techniques used in phlebotomy. The obtained blood samples may be further processed using standard techniques to obtain e.g. a serum sample, or a plasma sample. Advantageously, methods for obtaining biological fluid samples from a subject are typically low-invasive or non-invasive.

As mentioned, the present invention is based on the inventors' finding that a A4GS is a biomarker for AIH. Specifically, the inventors found that an increase in A4GS levels indicated that the subject has or is at risk of having AIH. A biomarker is a molecular, biological, or physical characteristic that can be measured or otherwise evaluated as an indicator of a normal biologic process, disease state, or response to a therapeutic intervention. A biomarker is differentially present if the mean or median level of the biomarker in the different groups is calculated to be statistically significant. Common tests for statistical significance include, among others, t-test (e.g., student t-test), ANOVA, Kruskal-Wallis, Wilcoxon, Mann-Whitney, Receiver Operating Characteristic (ROC curve), accuracy and odds ratio. Biomarkers, alone or in combination, provide measures of relative risk that a subject belongs to one phenotypic status or another. Therefore, they are useful as markers for disease (diagnostics), therapeutic effectiveness of a drug and drug toxicity. The biomarker of the invention is an “AIH-specific” biomarker, i.e., it is indicative of AIH, risk of AIH, and/or AIH progression. Biomarkers are detectable and/or measurable by any of a variety of methods such as biochemical and/or molecular assays.

In the context of the present disclosure “A4GS” refers to sialylation per galactose on tetraantennary glycans. Therefore, the phrase “level (or levels) of A4GS” refers to the amount of sialic acid moieties per galactose moiety of a tetraantennary glycan.

In general, a glycan is a saccharide, which includes a monosaccharide, a disaccharide or a trisaccharide; it can include an oligosaccharide or a polysaccharide. An oligosaccharide is an oligomeric saccharide that contains two or more saccharides. The structure of an oligosaccharide is typically characterized by particular identity, order, linkage positions (including branch points), and linkage stereochemistry (α, β) of the monomers, and as a result has a defined molecular weight and composition. An oligosaccharide typically contains about 2 to about 20 or more saccharide monomers. In a polysaccharide, the identity, order, linkage positions (including branch points) and/or linkage stereochemistry can vary from molecule to molecule. A protein with a glycan may be referred to as a glycoprotein. The glycan component of the glycoprotein can be N-linked or O-linked. An N-glycan is attached to a nitrogen atom, for example, at the side chain nitrogen atom of an asparagine amino acid within the peptide. An O-linked glycan is attached to an oxygen atom, for example at the side chain hydroxyl oxygen of a hydroxylysine, hydroxyproline, tyrosine, serine, or threonine amino acid within the peptide. “Glycosylation” refers to the covalent attachment of at least one saccharide moiety to a molecule. Glycosidic linkages include O-glycosidic linkages, N-glycosidic linkages, S-glycosidic linkages and C-glycosidic linkages. An O-glycosidic linkage is formed between the anomeric carbon (C1) of a saccharide and an oxygen atom of another molecule (such as another saccharide or a polypeptide), while an N-glycosidic linkage is formed between the anomeric carbon (C1) of a saccharide and a nitrogen atom of another molecule. Likewise, S-glycosidic linkages and C-glycosidic linkages involve a sulphur and carbon atom from another molecule, respectively. In addition, glycosidic linkages are classified according to the ring position of the carbon atoms participating in the bond. For example, a 1,4 glycosidic linkage is formed between the first carbon (C1) on a first saccharide and the fourth carbon (C4) on a second saccharide while a 1,6 glycosidic linkage is formed between the first carbon (C1) on a first saccharide and the sixth carbon (C6) on a second saccharide. Glycosidic linkages are further classified as α-glycosidic or β-glycosidic according to whether the substituent groups on the carbons flanking the oxygen in the saccharide are pointing in the same or opposite directions. The term “glycosylation” as used herein should be broadly construed so as to encompass the covalent linkage of any other carbohydrate moieties such as fucose and sialic acid, and as such includes fucosylation or sialylation. Most N-linked glycans share a common structure, referred to as a core, which typically contains three mannose, and two N-acetylglucosamine residues. The core may contain modifications such as sulfation or phosphorylation; the core may be intact or it may be truncated. Terminal modifications and core modifications of a glycan can include glycosylations. Core glycosylation refers to the addition of glycosyl moieties to a core N-acetylglucosamine and/or mannose. Core fucosylation refers to the addition of a fucose residue to the core N-acetylglucosamine. A glycan can be branched or unbranched. A complex type N-glycan is a glycan that contains at least one N-acetylglucosamine on each of the two mannose branches of the core. In a branched glycan, the monosaccharide at the branch point is covalently linked to two other saccharides at carbons other than C1. For example, a branch point monosaccharide may be linked to other monosaccharides at C4 and C6, in addition to being linked to another monosaccharide or to an amino acid at C1. A complex glycan may be, without limitation, biantennary (i.e. have two branches on the core structure), triantennary (i.e. have three branches on the core structure), or tetraantennary (i.e. have four branches on the core structure). One or more branch may be galactose-terminated. When the galactose moiety has a sialic acid moiety attached, it is said to be sialylated. When the antennae have one or more fucoses attached, it is said to be antenna fucosylated.

The methods provided herein refer to “determining” the level of A4GS. As would be clear to a person of skill in the art, the level of A4GS is typically “determined” by measuring the level of A4GS in the sample. The term “determining” can therefore be replaced with the term “measuring” or “determining by measuring” herein.

Conventional “determining” methods may include sending a clinical sample(s) to a commercial laboratory for measurement of the biomarker levels in the biological fluid sample, or the use of commercially available assay kits for measuring the biomarker levels in the biological fluid sample. Exemplary kits and suppliers will be apparent to a person of skill in the art. In various examples, biomarkers (in the context of present disclosure the biomarker being A4GS) may be determined, detected and/or quantified using mass spectrometry, high-performance liquid chromatography, capillary (gel) electrophoresis with laser induced fluoresces detection, hydrophilic interaction liquid chromatography (for example with fluorescence or UV detection), or an ELISA based assay (for example an ELISA based assay using glycan binding proteins such as lectins or antibodies). Exemplary methods for determining the levels of A4GS are also provided in the Examples section of the present specification. Merely by way of example,

A4GS may be calculated as follows: A4GS=((1/4*TA4S1+2/4*TA4S2+3/4*TA4S3+4/4*TA4S4)/TA4)/((1/4*TA4G1+2/4*TA4G2+3/4*TA4G3+4/4*TA4G4)/TA4).

Methods described herein comprise the step of comparing the level of A4GS to a reference level. It will be appreciated that the reference level may be derived from a control sample. A control sample is one that has normal levels of A4GS. Suitably, the control sample may be obtained from one or more subjects that do not have and/or are not at risk of having AIH. Suitably, the control sample may be the same type of sample as the test sample (i.e. the sample from the subject who is being tested to determine whether the subject has or is at risk of AIH). For example the control and test samples may be both plasma samples.

Suitably, the control sample may from a subject that has been matched to the test subject (i.e. for example is the same sex or age). However, surprisingly the present inventors have found that A4GS is neither confounded by the age and sex of the subject. Therefore, advantageously, the sample does not have to be matched.

The control sample may be assayed at the same time, before or after, separately or simultaneously with the test sample. The reference level that is used in the comparison with the test sample may be a value that is calculated as an average or median of more than one (e.g. two or more, five or more, ten or more, a group etc.) control samples. Alternatively, the control sample may be a sample that originated from (i.e. is a mix of) more than one (e.g. two or more, five or more, ten or more, a group etc.) individual that is not suffering and/or not believed to be at risk of AIH.

The reference level may be a predetermined reference level of A4GS. Such a predetermined reference level may be obtained from a reference database, which may be used to generate a pre-determined cut off value, i.e. a score that is statistically diagnostic or predictive of AIH.

In one example, the predetermined reference level is the average or median level of the biomarker in at least one individual not suffering or not at risk of AIH. The predetermined reference value may be calculated as the average or median, taken from a group or population of individuals that are not suffering and/or not at risk of AIH. For example, the predetermined reference value may be calculated as the average or median, taken from a group or population of individuals not suffering and/or not at risk of AIH.

Suitably, the reference level may be the level of A4GS in an individual or group of individuals that are believed to be of good overall health (i.e. with no diagnosed disorders and/or no symptoms indicative of a disorder).

In the context of the present disclosure, the term “change” refers to a statistically significant difference in the level of A4GS in the sample obtained from the test subject compared to the reference level. The difference (or change) may suitably be an increase in biomarker levels compared to the control sample or predetermined reference level. As mentioned elsewhere in the present disclosure, an increase in the level of A4GS as compared to reference level is indicative of the subject having or being at risk AIH. By same token “no change” refers to a statistically insignificant difference in the level of A4GS in the sample obtained from the test subject compared to the reference level. No change in A4GS may indicate that the subject does not have and/or is not at risk of having AIH.

The terms “increased”, “increase” or “up-regulated”, “higher” are all used herein to generally mean an increase by a statically significant amount; for the avoidance of any doubt, the terms “increased” or “increase” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 0.5-fold, or at least about a 1.0-fold, or at least about a 1.2-fold, or at least about a 1.5-fold, or at least about a 2-fold, or at least about a 3-fold, or at least about a 4-fold, or at least about a 5-fold or at least about a 10-fold increase, or any increase between 1.0-fold and 10-fold or greater as compared to a reference level.

The methods described herein can further comprise administering a treatment to the subject, when the subject has been determined to be in need of treatment for AIH. Suitably, the subject may be determined to be in need for treatment if the subject has been diagnosed to have or be at risk of AIH, or if the subject has been diagnosed to have AIH that is progressing.

Accordingly, in some aspects provided herein is a method for diagnosing AIH in a subject or predicting the risk of the AIH developing in a subject, and treating AIH in a subject, the method comprising the steps of:

a) determining the level of A4GS in a sample from the subject;

b) comparing the level of A4GS in the sample to a reference level;

c) identifying the subject as having AIH if the levels of A4GS in the sample are increased compared to the reference level; and

d) administering to the subject that has been identified as having AIH or being at risk of having AIH a treatment for AIH.

In some aspects, provided herein is a method of treating AIH in a subject, the method comprising the steps of administering an AIH treatment to a subject, wherein the subject has been diagnosed or determined to be at risk of AIH, or identified as having AIH by a method as described herein.

Suitably, the treatment may be prophylactic treatment or disease modifying treatment.

Suitably, the treatment may be an immunosuppressant, optionally the immunosuppressant may be prednisone, prednisolone, budesonide, azathioprine, mycophenolate mofetil, 6-thioguanine, 6-mercaptopurine, rituximab and/or tacrolimus.

Suitably, the treatment may be specific for AIH. By the treatment being specific for AIH it is meant that the treatment is typically not used in the treatment of other non-liver diseases and/or other liver diseases (such as PBC, PSC, NAFLD, viral hepatitis or alcoholic hepatitis).

As used herein, the terms “treat”, “treating”, and “treatment” are taken to include an intervention performed with the intention of preventing the development or altering the pathology of a condition, disorder or symptom (i.e. in this case AIH), for example preventing relapse. Accordingly, “treatment” refers to both therapeutic treatment and prophylactic or preventative measures, wherein the object is to prevent or slow down (lessen) the targeted condition, disorder or symptom. “Treatment” therefore may encompass a reduction, slowing or inhibition of the symptoms of AIH, for example of at least 5%, 10%, 20%, 30%, 40%, 50%, 60%, 70%, 80%, 90% or 100% when compared to the symptoms before treatment. Suitably, the treatment may be administered at a therapeutically effective amount. It will be appreciated that the therapeutically effective amount will vary depending on the type of treatment. Therapeutically effective amounts are well established and will be known to those skilled in the art.

The treatments described herein can be administered to the subject by any conventional route, for example orally (e.g. in for form of tablet or solution), orally, intravenously or intramuscularly. It will be appreciated that the preferable or appropriate route will depend upon the type of treatment, and optimal routes of administration will be known to those skilled in the art.

The methods disclosed herein may be combined with other tests for diagnosing AIH and disease monitoring. Whilst other tests useful in this context will be well known to those skilled in the art, merely by way of example they may include determining the levels of aspartate transaminase (AST), alanine transaminase (ALT) and/or IgG in the subject (or a sample from the subject), and/or by histological analysis of a liver biopsy.

In a further aspect, the present invention also provides a kit for use in the methods disclosed herein. The kits may include reagents suitable for determining levels of an analyte in a test sample (e.g., reagents suitable for determining levels of the biomarker disclosed herein).

Suitably, the kit comprises a detectably labelled agent that specifically binds to A4GS.

The kit may further comprise a detectably labelled agent that specifically binds to aspartate AST, ALT and/or IgG.

Optionally, the kit may contain one or more control samples or references. Typically, a comparison between the levels of the biomarkers in the subject and levels of the biomarkers in the control samples is indicative of a clinical status (e.g., diagnosis of AIH or risk of developing AIH etc.). Also, the kits, in some cases, will include written information (indicia) providing a reference (e.g., pre-determined values), wherein a comparison between the levels of the biomarkers in the subject and the reference (pre-determined values) is indicative of a clinical status. In some cases, the kits comprise software useful for comparing biomarker levels or occurrences with a reference (e.g., a prediction model). Usually the software will be provided in a computer readable format such as a compact disc, but it also may be available for downloading via the internet. However, the kits are not so limited and other variations with will apparent to one of ordinary skill in the art.

The components of the kit may be housed in a container that is suitable for transportation.

The term “detectably labelled agent” refers to a binding partner that interacts (i.e. binds) specifically with the biomarker of interest A4GS or hybrid-type glycans and is also capable of being detected e.g. directly (such as via a fluorescent tag) or indirectly (such as via a labelled secondary antibody). The detectably labelled agent is therefore a selective binding partner for the biomarker of interest (and does not substantially bind to other proteins). Selective binding partners may include antibodies that selectively bind to one of the biomarker of interest.

As used herein, “specifically binds to A4GS” means that under certain conditions the binding partner that “specifically binds to A4GS” will selectively bind A4GS will not bind in a significant amount to other peptides, proteins, and/or protein modifications, including other glycans. Thus the binding partner may bind to A4GS with at least 10, 20, 30, 40, 50, 60, 70, 80, 90 or 100 fold more affinity than it binds other peptides, proteins, and/or protein modifications, including other glycosylations.

In some examples the kits include the detectably labelled agent(s) on a continuous (e.g. solid) surface, such as a lateral flow surface. Alternatively, in examples comprising more than one detectably labelled agent, the detectably labelled agent(s) may be located in distinct (i.e. spatially separate) zones on a (e.g. solid) surface, such as a multiwall micro-titre plate (e.g. for an ELISA assay). Other appropriate surfaces and containers that are well known in the art may also form part of the kits described herein.

In one example, the kit further comprises one or more reagents for detecting the detectably labelled agent. Suitable reagents are well known in the art and include but are not limited to standard reagents and buffers required to perform any one of the appropriate detection methods that may be used (and are well known in the art). In one example, the kit comprises one or more of the following: a multi-well plate, ball bearing(s), extraction buffer, extraction bottle and a lateral flow device lateral flow device.

Provided herein is also the use of a A4GS as a biomarker for AIH. Suitably, the biomarker is a biological fluid (e.g. plasma, whole blood and/or urine) biomarker for AIH.

Aspects of the invention are demonstrated by the following non-limiting examples.

### Examples

1 Materials and Methods

1.1 Study Design

In this cross-sectional study, samples were obtained from the Leiden University Medical Center biobank. All AIH patients were diagnosed according to the simplified² or revised³ AIH criteria and samples between 2004 and 2020, before or during treatment, were included. Patients with variant syndromes were excluded. Matched on age and sex to 66 AIH patients (with overall 214 samples) 60 healthy controls were included.

Furthermore, as control groups, 31 PSC, 10 PBC and 30 NAFLD were included, as well as 15 patients with viral or alcoholic hepatitis without cirrhosis, 29 patients with compensated cirrhosis and 30 patients with decompensated cirrhosis. Baseline and longitudinal characteristics of the cohort can be found in Table 1. The study protocol was approved a priori by the local ethical committee (B19.071). Informed consent was obtained from all patients and healthy controls. The study complied to the latest version of the Declaration of Helsinki.

Patients who were not treated at the moment of sampling were defined as “before treatment”. This could be at diagnosis or at a relapse after cessation of immunosuppressive medication. Patients in complete biochemical remission, defined as normalization of AST, ALT and IgG were classified as “remission”, while “no remission” included patients with an incomplete response, loss of remission or relapse during treatment. Incomplete response was defined as increases in of AST, ALT and IgG without normalization. Loss of remission was defined as AST or ALT between 1-3× upper limit of normal while relapse was defined as AST or ALT>3 times the upper limit of normal. Presence of cirrhosis was based on liver histology. If liver histology was not available, liver elastography or liver ultrasound were used. Decompensated liver cirrhosis was defined as presence of ascites, varices bleeding, hepatocellular carcinoma, hepatorenal or hepatopulmonary syndrome.

TABLE 1
Baseline and longitudinal characteristics of patients and healthy controls enrolled
in the cohort. Median and interquartile ranges are shown unless indicated otherwise.

| | | | | | | | | | | | Viral and alcoholic hepatitis | | | | | |
|---|---|---|---|---|---|---|---|---|---|---|---|---|---|---|---|---|
| | | | | | | | | | | | Without | | Compensated | | Decompensated | |
| | AIH | | HC | | PBC | | PSC | | NAFLD | | cirrhosis | | cirrhosis | | cirrhosis | |
| Patients | 66 | | 60 | | 10 | | 31 | | 30 | | 15 | | 29 | | 30 | |
| Number of | 214 | [3.2] | 60 | [1] | 10 | [1] | 31 | [1] | 30 | [1] | 15 | [1] | 29 | [1] | 30 | [1] |
| samples | | | | | | | | | | | | | | | | |
| [mean per | | | | | | | | | | | | | | | | |
| patient] | | | | | | | | | | | | | | | | |
| Female | 47 | [71] | 43 | [72] | 9 | [90] | 10 | [32] | 17 | [57] | 6 | [40] | 5 | [17] | 9 | [30] |
| gender [%] | | | | | | | | | | | | | | | | |
| Age female | 50 | [27-62] | 49 | [32-63] | 52 | [50-53] | 48 | [36-66] | 60 | [49-67] | 55 | [51-55] | 47 | [45-57] | 59 | [57-63] |
| Age male | 56 | [32-63] | 55 | [32-63] | 57 | [—] | 50 | [38-61] | 43 | [29-62] | 59 | [48-61] | 53 | [50-57] | 56 | [47-59] |
| Cirrhosis at | | | | | | | | | | | | | | | | |
| first sample | | | | | | | | | | | | | | | | |
| [%] | | | | | | | | | | | | | | | | |
| No cirrhosis | 39 | [59] | | | 7 | [70] | 19 | [61] | 26 | [87] | 15 | [100] | 0 | [0] | 0 | [0] |
| Compensated | 22 | [33] | | | 3 | [30] | 11 | [35] | 3 | [10] | 0 | [0] | 29 | [100] | 0 | [0] |
| cirrhosis | | | | | | | | | | | | | | | | |
| Decompensated | 5 | [7.6] | | | 0 | [0] | 1 | [3] | 1 | [3] | 0 | [0] | 0 | [0] | 30 | [100] |
| cirrhosis | | | | | | | | | | | | | | | | |
| Laboratory | | | | | | | | | | | | | | | | |
| values | | | | | | | | | | | | | | | | |
| AST (IU/L) | 37 | [26-59] | | | 50 | [35-54] | 50 | [38-92] | 29 | [24-42] | 30 | [17-37] | 87 | [45-116] | 61 | [49-99] |
| ALT (IU/L) | 38 | [23-67] | | | 38 | [28-77] | 54 | [37-106] | 41 | [36-71] | 25 | [21-57] | 47 | [26-70] | 30 | [25-40] |
| ALP (IU/L) | 88 | [64-120] | | | 277 | [158-433] | 214 | [148-319] | 79 | [70-95] | 107 | [94-125] | 123 | [95-145] | 117 | [104-166] |
| GGT (IU/L) | 58 | [25-163] | | | 147 | [58-571] | 162 | [86-256] | 44 | [29-60] | 27 | [21-69] | 60 | [38-93] | 66 | [30-96] |
| IgG (g/L) | 12 | [10-17] | | | | | | | | | | | | | | |
| Treatment | | | | | | | | | | | | | | | | |
| response at | | | | | | | | | | | | | | | | |
| first sample | | | | | | | | | | | | | | | | |
| [at all | | | | | | | | | | | | | | | | |
| timepoints] | | | | | | | | | | | | | | | | |
| No treatment | 14 | [19] | | | | | | | | | | | | | | |
| No complete | 31 | [115] | | | | | | | | | | | | | | |
| biochemical | | | | | | | | | | | | | | | | |
| remission | | | | | | | | | | | | | | | | |
| Complete | 21 | [75] | | | | | | | | | | | | | | |
| biochemical | | | | | | | | | | | | | | | | |
| remission | | | | | | | | | | | | | | | | |
| Medication at | | | | | | | | | | | | | | | | |
| first timepoint | | | | | | | | | | | | | | | | |
| [at all | | | | | | | | | | | | | | | | |
| timepoints] | | | | | | | | | | | | | | | | |
| No medication | 14 | [19] | | | | | | | | | | | | | | |
| Steroid based | 43 | [136] | | | | | | | | | | | | | | |
| treatment | | | | | | | | | | | | | | | | |
| Steroid free | 9 | [58] | | | | | | | | | | | | | | |
| treatment | | | | | | | | | | | | | | | | |

AIH: autoimmune hepatitis;
NAFLD: non-alcoholic fatty liver disease;
PBC: primary biliary cholangitis;
PBC: primary sclerosing cholangitis;
WC: without cirrhosis;
CC: compensated cirrhosis;
DC: decompensated cirrhosis;
AST: aspartate aminotransferase;
ALT: alanine aminotransferase;
GGT: gamma-glutamyl transferase.

1.2 Mass Spectrometry Glycomics and Data Processing

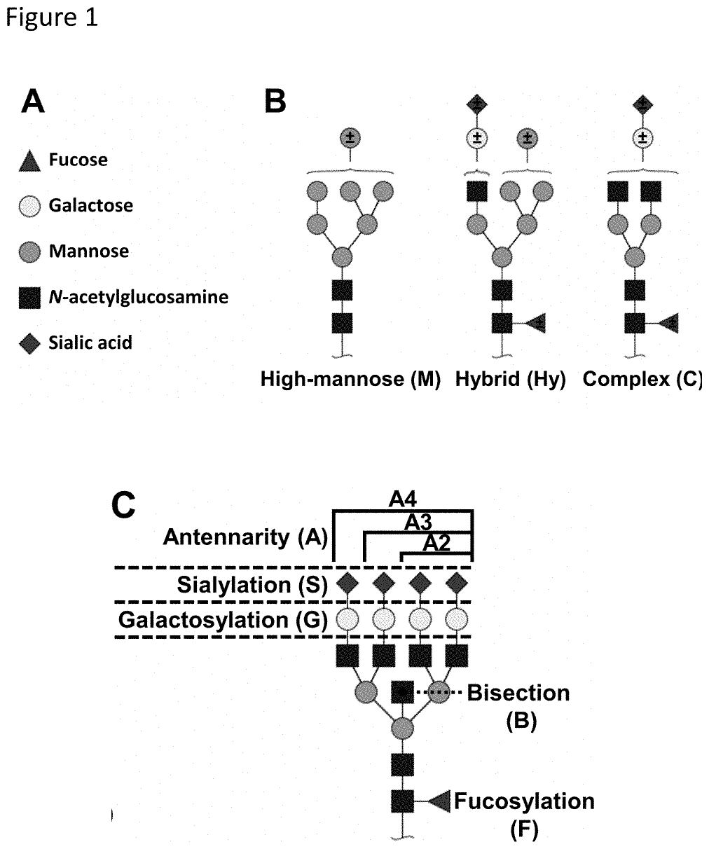

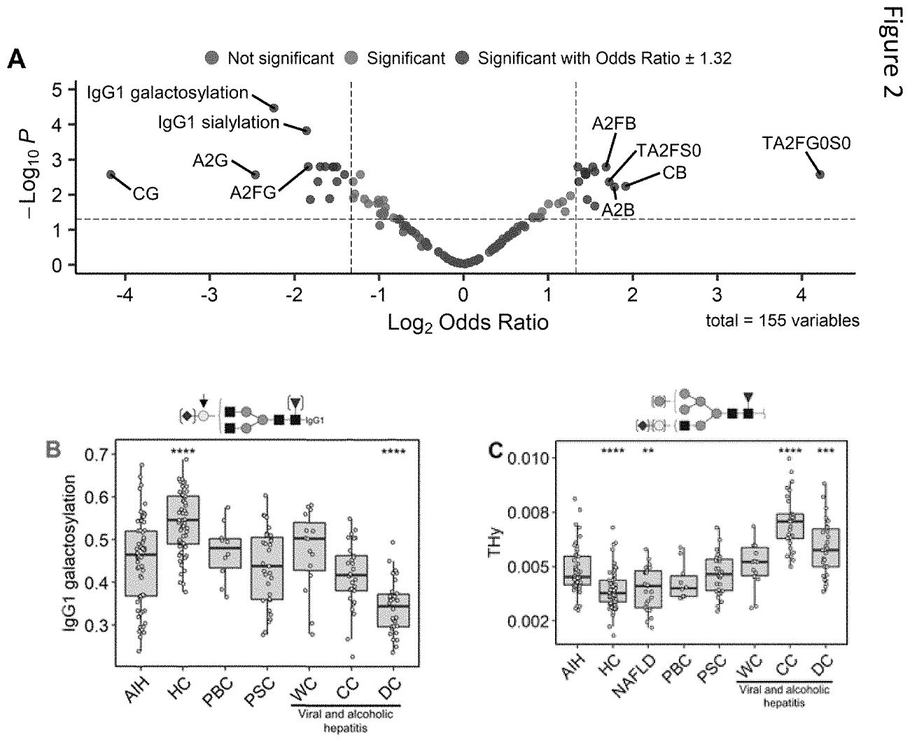

The total plasma N-glycome of plasma samples was analysed by matrix assisted laser desorption ionization-Fourier-transform ion cyclotron resonance-mass spectrometry (MALDI-FTICR-MS) after linkage-specific sialic acid derivatization, as described²⁰. Release of N-glycans from plasma proteins and linkage-specific chemical sialic acid derivatization was performed as previously described in similar high-throughput, robotized workflow, using 2 μL of plasma/serum for the release^{20,45,46}. For MALDI-FTICR-MS measurement, 1 uL sDHB matrix was topped by 1 uL HILIC-purified sample and left to dry by air^{20,45,46}. The measurement was performed on a 15 T Bruker SolariX XR FTICR mass spectrometer equipped with a ParaCell, a Smartbeam-II laser and a Combisource (Bruker Daltonics, Bremen, Germany) in positive ionization mode^{20,46}. Calibration was performed with Peptide Calibration Mix II (Bruker Daltonics). IgG Fc glycosylation was analysed at the glycopeptide level with nano liquid chromatography coupled to MS (nLC-MS), as reported before²¹. After initial data pre-processing including data quality control, similarly to foregoing reports^{10,22}, the relative abundances of individual glycans as well as of derived glycosylation traits (e.g. ratios of glycan abundances reflecting specific enzymatic biosynthetic steps) were calculated (FIG. 1). A4GS is a calculated trait based on the relative abundances of specific N-glycans. It describes the relative abundance of sialylation on tetra-antennary N-glycans as a fraction of the relative abundance of galactosylation on tetra-antennary N-glycans. A4GS may be calculated as follows, where any N-glycan that falls in the described categories may be used:

A4GS=((1/4*TA4S1+2/4*TA4S2+3/4*TA4S3+4/4*TA4S4)/TA4)/((1/4*TA4G1+2/4*TA4G2+3/4*TA4G3+4/4*TA4G4)/TA4); where TA4 indicates the total sum of all tetra-antennary N-glycans, irrespective of their further elongation. Substitutes S1-S4 and G1-G4 indicate the subsets of this sum that contain glycans with one to four sialic acids (S) or galactoses (G), respectively.

In the present examples, A4GS was calculated using the following formula:

A4GS=((0/4*(0)+1/4*(H6N6F1E1+H7N6E1)+2/4*(H7N6E2)+3/4*(H7N6L2E1+H7N6L1E2+H7N6E3+H7N6F1L2E1+H7N6F1L1E2)+4/4*(H7N6L3E1+H7N6L2E2+H7N6L1E3+H7N6F1L3E1+H7N6F1L2E2+H7N6F2L3E1+H7N6F2L2E2))/(H6N6F1E1+H7N6E1+H7N6E2+H7N6L2E1+H7N6L1E2+H7N6E3+H7N6F1L2E1+H7N6F1L1E2+H7N6L3E1+H7N6L2E2+H7N6L1E3+H7N6F1L3E1+H7N6F1L2E2+H7N6F2L3E1+H7N6F2L2E2))/((0/4*(0)+1/4*(0)+2/4*(0)+3/4*(H6N6F1E1)+4/4*(H7N6E1+H7N6E2+H7N6L2E1+H7N6L1E2+H7N6E3+H7N6F1L2E1+H7N6F1L1E2+H7N6L3E1+H7N6L2E2+H7N6L1E3+H7N6F1L3E1+H7N6F1L2E2+H7N6F2L3E1+H7N6F2L2E2))/(H6N6F1E1+H7N6E1+H7N6E2+H7N6L2E1+H7N6L1E2+H7N6E3+H7N6F1L2E1+H7N6F1L1E2+H7N6L3E1+H7N6L2E2+H7N6L1E3+H7N6F1L3E1+H7N6F1L2E2+H7N6F2L3E1+H7N6F2L2E2)); wherein H=hexose; N=N-acetylhexosamine; F=deoxyhexose (fucose); L=lactonized N-acetylneuraminic acid (α2,3-linked); E=ethyl esterified N-acetylneuraminic acid (α2,6-linked).

MALDI-FTICR-MS raw spectra were converted into xy files, whereas mzXML files were generated from the nanoLC-MS raw spectra. Extraction of these raw data was performed using in-house developed software MassyTools⁴⁷ and LaCyTools⁴⁸, respectively. Chromatograms were aligned based on average retention time and exact mass of five highest abundant glycoforms in each IgG cluster. For the targeted extraction of glycan and glycopeptide peaks, analyte lists were created based on manual annotation of summed mass spectra per disease group. The assignment of glycoforms was based on exact mass and previous reports^{20,23,49,50}. For MALDI-FTICR-MS data, the 1+ charge state was used for extraction, whilst the 2+ and 3+ charge states were used for nanoLC-MS data extraction. Signals were integrated by covering minimum 95% of the area of the isotopic envelope of glycan and glycopeptide peaks. Serum samples (considering 26 AIH and 2 PSC patients) were excluded from the TPNG analysis, because serum, unlike plasma, is depleted from blood clotting factors, such as the abundant glycoprotein fibrinogen, making comparison of the two inherently biased. An analyte was included in the final data analysis if its signal-to-noise was above 27, its isotopic pattern did not deviate more than 25% from the theoretical one, and if its mass error was within a ±20 parts per million range. Additionally, the same analyte (glycan or glycopeptide for TPNG and IgG, respectively) had to be present in at least 1 out of 4 spectra (25%) in each disease group for inclusion to the final data analysis. The relative intensity values of glycan compositions that passed quality criteria were calculated by normalizing to the sum of their total areas in case of TPNG, and per subclass for IgG.

1.3 Statistical Analysis

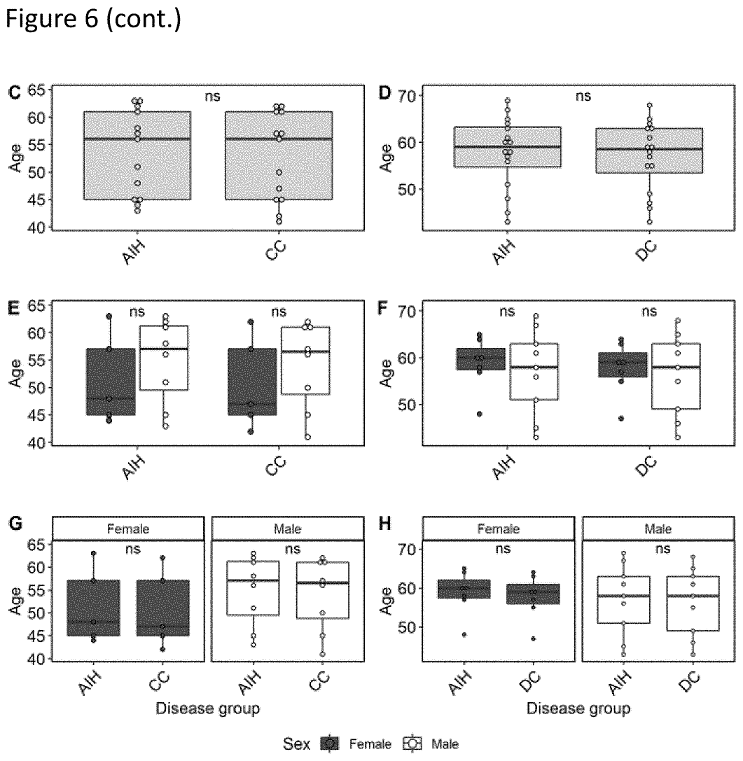

A logistic regression model on standardized data (subtraction of the mean and division by the SD) including age, sex and their interaction as co-variates was used to study the associations between glycosylation of healthy controls (HC) and AIH patients (HC=0; AIH=1) (Table 2). Glycosylation traits that were significantly different based on the logistic regression results were compared between disease groups using a Wilcoxon rank-sum test (FIG. 6-9), while a Wilcoxon-signed-rank test was used to compare longitudinal timepoints corresponding to the same patient. To account for multiple testing, during the evaluation of statistical significance per statistical question (Table 2, Benjamini-Hochberg procedure with a false discovery rate (FDR) of 5% was used.

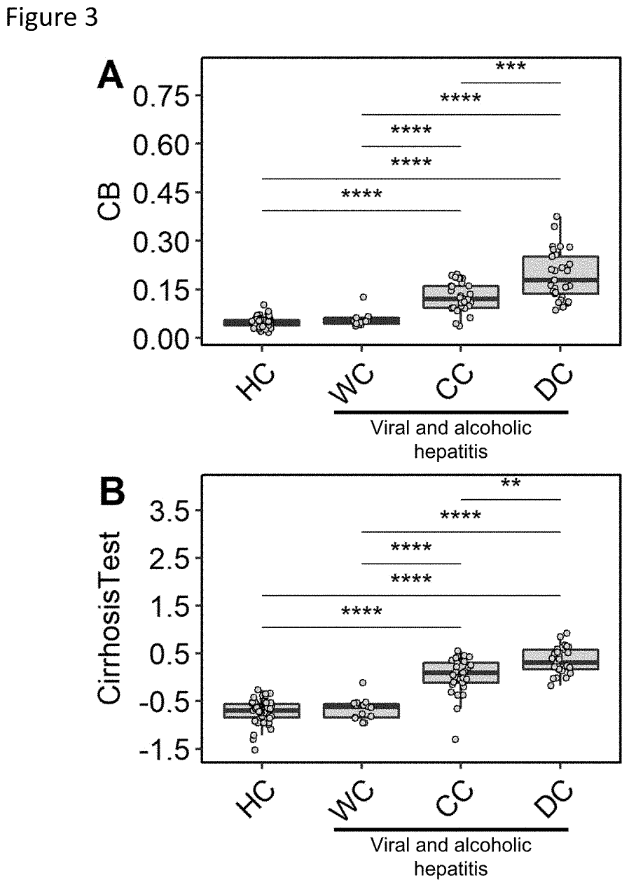

TABLE 2
Associations between plasma and IgG N-glycan traits and AIH as compared to healthy controls.
Logistic regression was performed between AIH (1) and HC (0), including age, sex and their
interaction as co-variates. Only significant associations that passed the set log₂ odds
ratio threshold (FIG. 3A) are shown. To account for multiple testing, p-values have
been corrected by the Benjamini-Hochberg procedure using a 5% FDR.

| | | p-value (age | Odds ratio [95% CI] | | | |
|---|---|---|---|---|---|---|
| | | and sex | (age and sex | | Beta | Standard |
| Derived traits | Description | corrected) | corrected) | | coefficient | error |
| Glycan type | | | | | | |
| MHy | High mannose to | 1.62E−03 | 0.34 | [0.2-0.56] | −1.06 | 0.27 |
| | hybrid-type ratio | | | | | |
| THy | Total hybrid within | 2.31E−03 | 2.73 | [1.67-4.89] | 1.01 | 0.27 |
| | total | | | | | |
| TA2FG0S0 | Total fucosylated | 2.72E−03 | 18.55 | [4.7-112.74] | 2.92 | 0.81 |
| | nongalactosylated | | | | | |
| | nonsialylated A2 | | | | | |
| TA2FS0 | Total fucosylated | 4.41E−03 | 3.3 | [1.76-7.09] | 1.19 | 0.35 |
| | nonsialylated A2 | | | | | |
| Bisection | | | | | | |
| CB | Of complex-type | 5.88E−03 | 3.78 | [1.88-9.35] | 1.33 | 0.41 |
| A2B | Of A2 | 6.10E−03 | 3.45 | [1.79-7.99] | 1.24 | 0.38 |
| A2SB | Of sialylated A2 | 1.40E−02 | 2.76 | [1.52-5.89] | 1.01 | 0.34 |
| A2FB | Of fucosylated A2 | 1.63E−03 | 3.21 | [1.87-6.16] | 1.17 | 0.30 |
| A2FSB | Of fucosylated | 2.72E−03 | 2.72 | [1.65-4.96] | 1.00 | 0.28 |
| | sialylated A2 | | | | | |
| A2S0B | Of nonsialylated A2 | 2.23E−03 | 2.93 | [1.74-5.39] | 1.08 | 0.29 |
| A2F0S0B | Of nonfucosylated | 4.28E−03 | 2.57 | [1.55-4.65] | 0.94 | 0.28 |
| | nonsialylated A2 | | | | | |
| A2F0B | Of nonfucosylated | 2.12E−02 | 2.93 | [1.49-6.89] | 1.08 | 0.39 |
| | A2 | | | | | |
| A2FS0B | Of nonfucosylated | 2.72E−03 | 2.71 | [1.64-4.86] | 1.00 | 0.28 |
| | sialylated A2 | | | | | |
| Galactosylation | | | | | | |
| CG | Of complex-type | 2.72E−03 | 0.06 | [0.01-0.22] | −1.19 | 0.35 |
| A2G | Of A2 | 2.77E−03 | 0.18 | [0.06-0.42] | −1.70 | 0.48 |
| A2FG | Of fucosylated A2 | 1.60E−03 | 0.28 | [0.14-0.49] | −1.27 | 0.31 |
| A2SG | Of sialylated A2 | 1.31E−02 | 0.33 | [0.15-0.63] | −1.10 | 0.37 |
| A2S0G | Of nonsialylated A2 | 1.60E−03 | 0.31 | [0.17-0.52] | −1.17 | 0.29 |
| A2FSG | Of fucosylated | 2.72E−03 | 0.38 | [0.21-0.62] | −0.97 | 0.27 |
| | sialylated A2 | | | | | |
| A2FS0G | Of fucosylated | 1.60E−03 | 0.32 | [0.18-0.54] | −1.13 | 0.28 |
| | nonsialylated A2 | | | | | |
| A2F0S0G | Of nonfucosylated | 4.28E−03 | 0.35 | [0.18-0.61] | −1.04 | 0.30 |
| | nonsialylated A2 | | | | | |
| Sialylation | | | | | | |
| CS | Of complex-type | 4.28E−03 | 0.3 | [0.14-0.57] | −1.19 | 0.35 |
| A4S | Of A4 | 2.25E−03 | 2.7 | [1.66-4.74] | 0.99 | 0.27 |
| A4GS | Per galactose on | 1.63E−03 | 2.88 | [1.75-5.16] | 1.06 | 0.27 |
| | A4 | | | | | |
| A4F0GS | Per galactose on | 2.72E−03 | 2.73 | [1.64-4.92] | 1.00 | 0.28 |
| | nonfucosylated A4 | | | | | |
| IgG glycosylation | | | | | | |
| derived traits | | | | | | |
| IgG1 bisection | IgG1 bisection | 1.63E−03 | 2.55 | [1.63-4.22] | 0.94 | 0.24 |
| IgG1 | IgG1 | 3.47E−05 | 0.21 | [0.11-0.37] | −1.55 | 0.30 |
| galactosylation | galactosylation | | | | | |
| IgG1 sialylation | IgG1 sialylation | 1.52E−04 | 0.28 | [0.16-0.45] | −1.29 | 0.27 |
| IgG1 antennary | IgG1 antennary | 1.62E−03 | 0.34 | [0.19-0.56] | −1.07 | 0.27 |
| fucosylation | fucosylation | | | | | |
| IgG2/3 | IgG2/3 | 1.62E−03 | 0.35 | [0.2-0.58] | −1.04 | 0.26 |
| galactosylation | galactosylation | | | | | |

CI: confidence interval.

2 Results

TPNG and IgG glycosylation were analysed by MS, resulting in the relative quantification of 81 glycans for TPNG. For IgG1, IgG2/3 and IgG4 a total of 15, 12 and 13 glycans were quantified, respectively. The quantified glycoforms were summarized in derived glycosylation traits (FIG. 1). For the exclusively diantennary IgG glycans²³, these traits encompassed fucosylation, bisection, galactosylation and sialylation (FIG. 1). For the TPNG glycans traits, also antennarity and N-glycan type were included (FIG. 1). The identified glycoforms were characteristic for plasma proteins and IgG in agreement with literature^{20,23,24}.

2.1 Plasma N-Glycan and IgG-Specific Glycan Traits Associate with Autoimmune Hepatitis

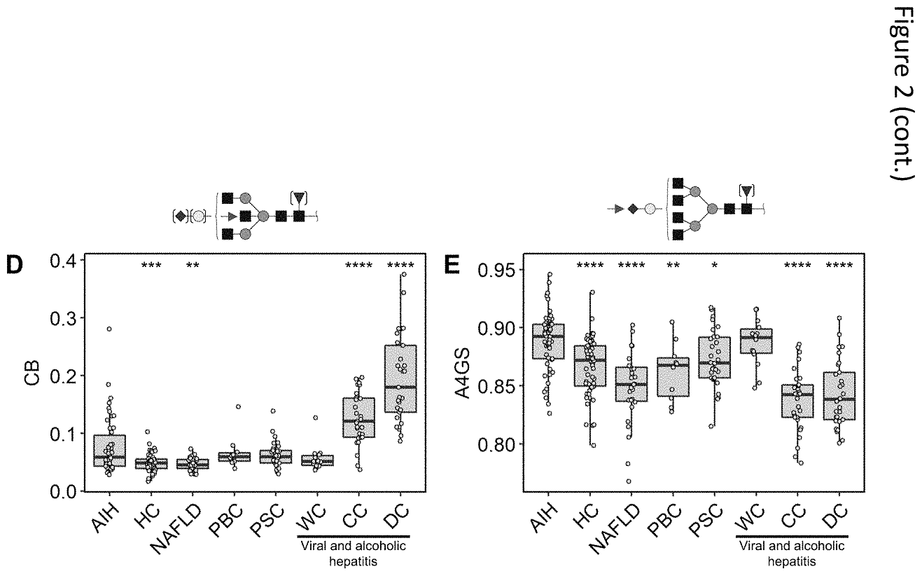

Comparing the derived glycan traits between AIH and healthy controls, 30 significant associations were found (FIG. 2A, Table 2). From these associations, the five glycosylation traits with the largest negative effect size (odds ratio (OR) between 0.28 and 0.06, corrected p-value (hereafter p-value)<3×10⁻³) were traits describing galactosylation levels in TPNG (A2G, CG, A2FG) as well as IgG1-specific galactosylation and sialylation (FIG. 2A, Table 2). The strongest positive effect size (OR between 3.21 and 18.55, p-value<7×10⁻³) was found for traits describing bisection levels in TPNG (A2B, CB, A2FB) as well as traits indicating the absence of galactoses and sialic acids on diantennary TPNG glycans (TA2FS0 and TA2FG0S0) (FIG. 2A, Table 2). All 30 glycosylation traits showing a difference between AIH and healthy controls were then further investigated in patients suffering from NAFLD, PBC, PSC and viral or alcoholic hepatitis.

2.2 TPNG Sialylation Per Galactose on Tetraantennary Glycans

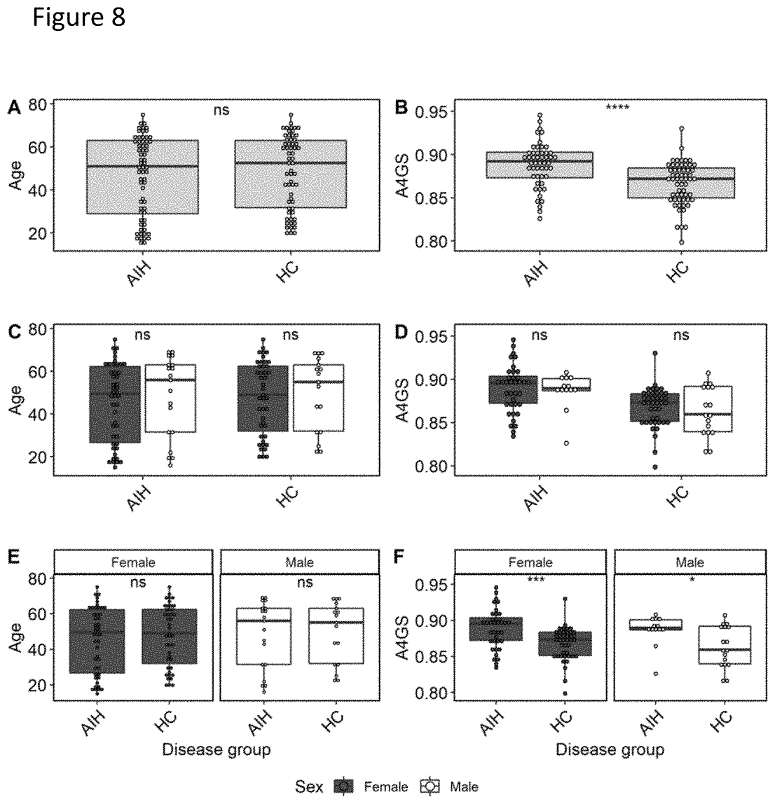

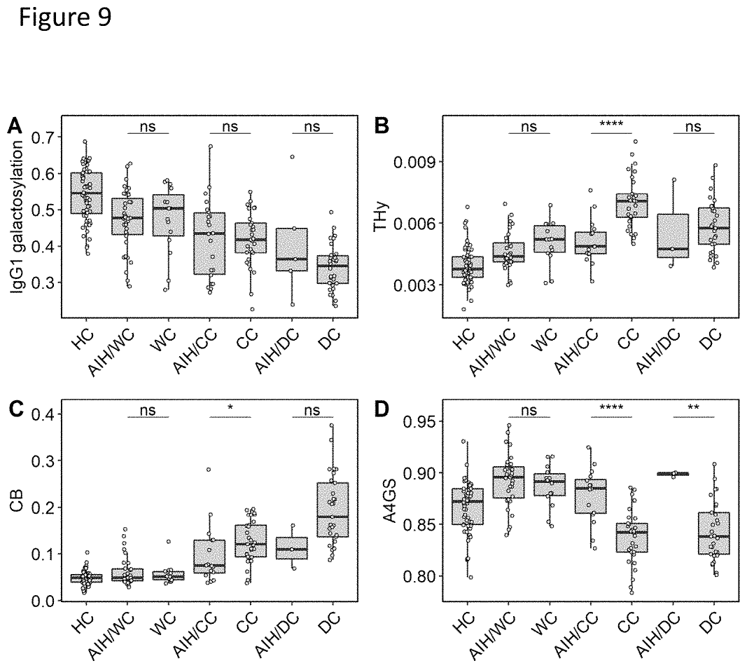

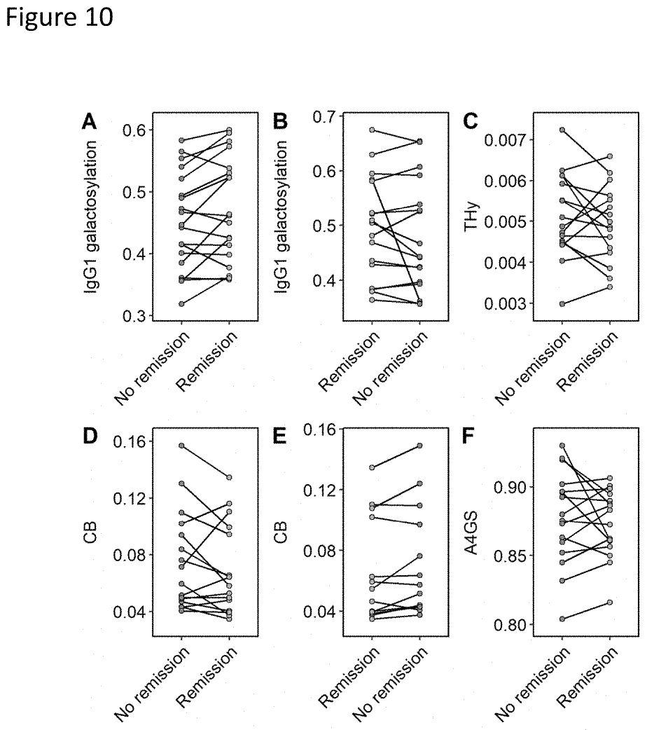

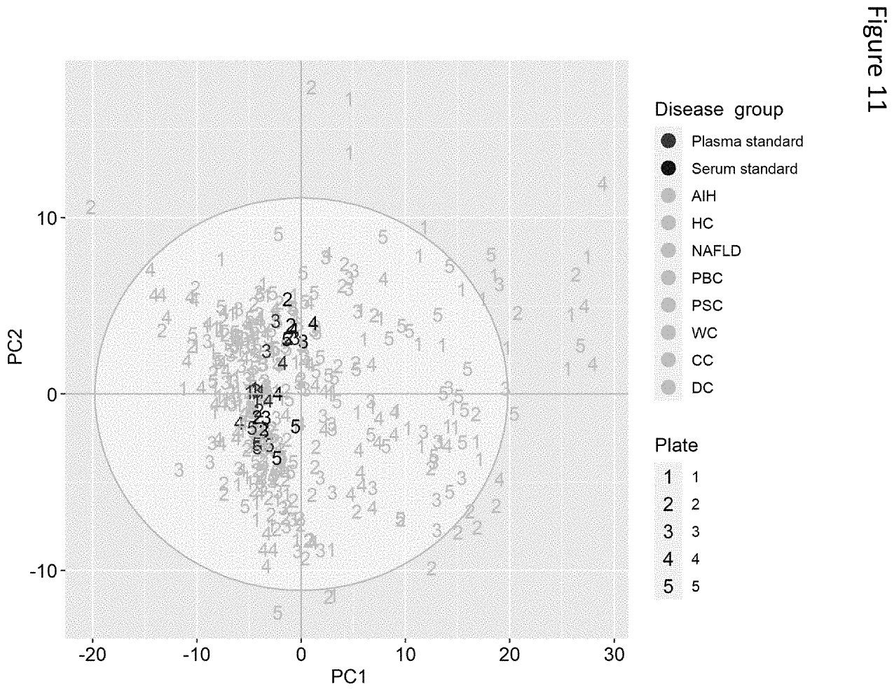

Interestingly, we observed a higher sialylation per galactose on tetraantennary glycans (A4GS) as an AIH-specific plasma N-glycan signature (OR: 2.88, 95% confidence interval (CI) [1.75-5.16], p-value: 1.63×10⁻³). A4GS was not only different in AIH patients when compared to HC, but also compared to all other disease groups in the cohort, except for patients with viral hepatitis without cirrhosis (WC) (FIG. 2E). Importantly, A4GS is neither confounded by the age and sex of the patient, nor by cirrhosis occurrence or severity (FIG. 8-9).

2.3 TPNG Bisection

TPNG bisection (CB) associated positively with AIH as compared to HC as well as to NAFLD patients. Because bisection is a known marker for cirrhosis, the degree of TPNG bisection was monitored in alcoholic and viral hepatitis patients, who were stratified based on cirrhosis severity (FIG. 3, Table 1). CB showed no difference between healthy individuals and viral hepatitis patients without cirrhosis (FIG. 3A). Patients suffering from compensated or decompensated cirrhosis (CC and DC, respectively) featured increased levels of bisection (CC fold change (FC): 2.4730, p-value: 6.90×10⁻¹¹; DC FC: 3.6734, p-value: 2.60×10⁻¹³), with a more pronounced effect for DC (FIG. 3A 8). Similar observations were made when we used our data to simulate the GlycoCirrhoTest^{7,8} which measures the bisection of diantennary glycans, relatively to triantennary glycans (FIG. 3B). The reported cirrhosis effect was also seen within the AIH patients (FIG. 9).

2.4 IgG Galactosylation

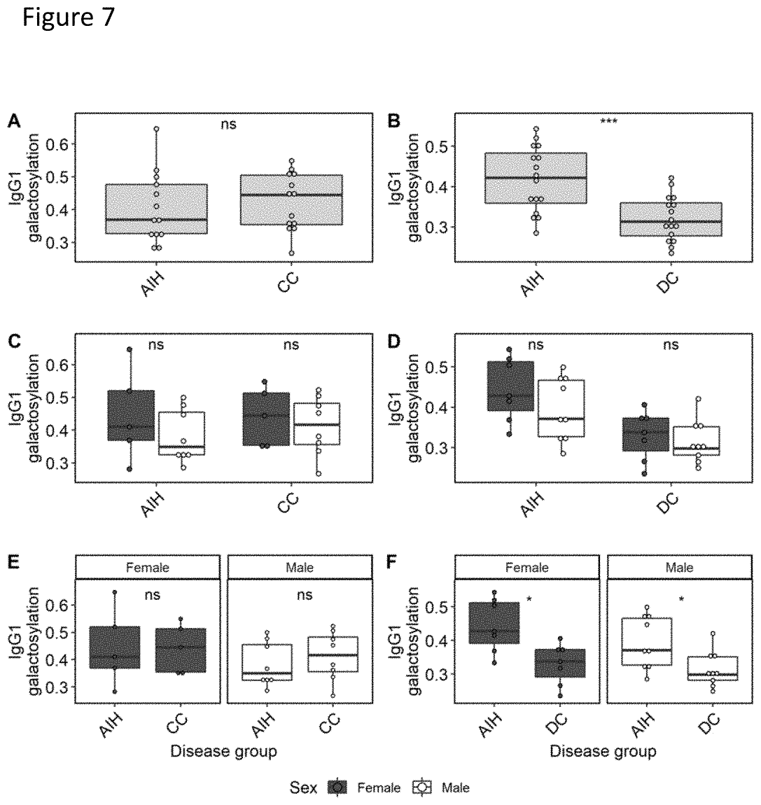

Although IgG1 galactosylation was decreased in AIH patients as compared to HC, this was not unique for AIH but occurred in other disease groups (FIG. 2B). Patients suffering from hepatitis with DC featured an even lower IgG galactosylation than the AIH patients (FIG. 2B). A negative relation between IgG1 galactosylation and cirrhosis severity was observed for the hepatitis as well as the AIH patients (FIG. 2B, FIG. 7, 9).

2.5 TPNG Hybrid-Type Glycans

The total level of hybrid-type glycans (THy) in AIH was higher as compared to HC but had no discriminative power towards the other liver diseases. THy showed a positive association with the occurrence of cirrhosis, both for the hepatitis and the AIH patients (FIG. 2C, Table 2).

2.6 Plasma N-Glycan and IgG-Specific Glycan Traits Associate with Treatment and Disease Activity in AIH

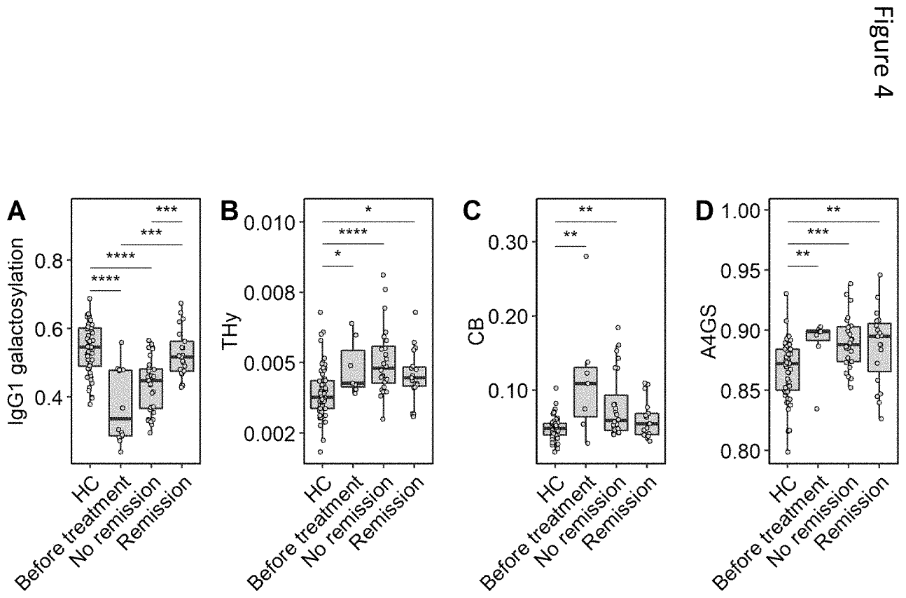

To investigate glycosylation features associated with disease activity, we separated the first available sample per patient based on treatment response (i.e., before treatment, no remission or complete biochemical remission) (Table 1). IgG1 galactosylation of patients with active disease (i.e., before treatment or not in remission) was lower as compared to HC (FC: 0.6158, p-value: 4.2×10⁻⁶), while patients in remission did not show an IgG galactosylation effect as compared to HC (FIG. 4A). The same was observed for CB, which was significantly different between patients with an active disease as compared to HC, while no difference was seen for patients in remission. However, when following these traits longitudinally in patients developing from remission to no remission or vice versa, no differences in IgG galactosylation or CB were observed. Within the treated patients, a slightly higher level of IgG galactosylation was found for the steroid treated group, as compared to non-treated, while other comparisons did not show differences.

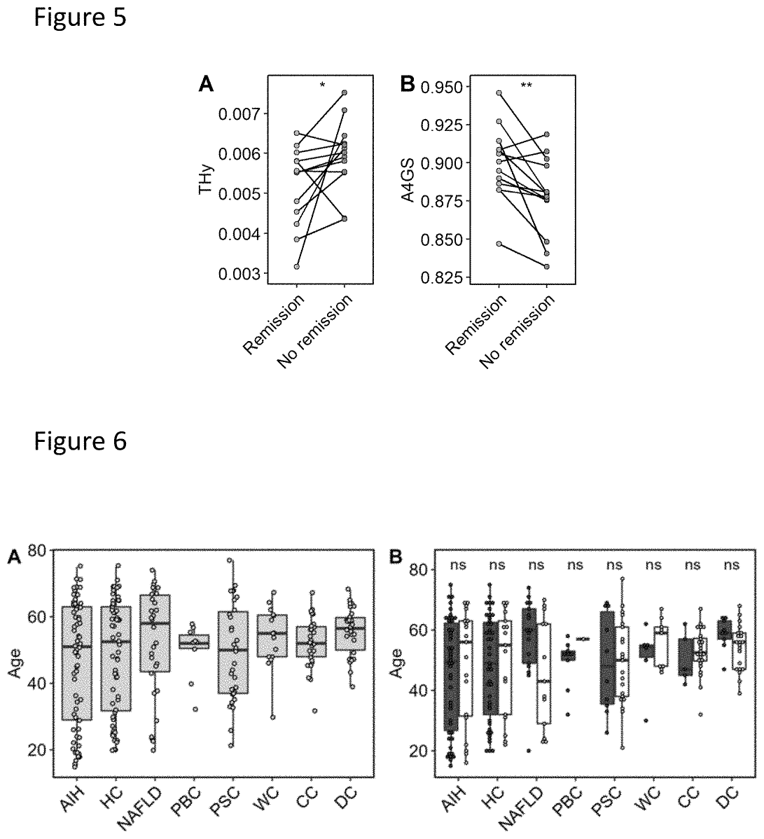

The traits THy and A4GS were consistently higher in AIH patients as compared to healthy controls, independent of their treatment status (FIG. 4B, D). Interestingly, a difference was observed for both traits in a longitudinal manner in patients developing from remission to no remission, with THy increasing, while A4GS decreased (FIG. 5). The reverse effect (i.e., for patients developing from no remission to remission) was not observed (FIG. 5).

3 Discussion

In this cross-sectional cohort study, elevated A4GS was found as a unique marker in patients with AIH compared to healthy controls and other liver diseases. Liver inflammation and cirrhosis were shown to be important confounders influencing glycosylation patterns, but A4GS was increased in AIH patients independently of these factors. By the inventors' knowledge this is the first study to extensively investigate glycosylation in AIH compared to healthy controls and other liver diseases and the findings offer opportunities to facilitate the non-invasive and accurate diagnosis of AIH.

Currently, diagnosis of AIH is based on the revised² or the simplified³ AIH criteria. Although these clinical criteria exist, in practice it can be challenging to distinguish AIH from PBC, PSC and NAFLD, and often a liver biopsy is needed. To reduce the invasive and risky need of liver biopsies, blood-derived markers to distinguish the diseases are desirable. The inventors used an exploratory, MALDI-FTICR-MS based approach for studying plasma N-glycosylation, with high glycoform resolution and the ability to study sialylation and its linkages²⁰. The approach identified high A4GS as AIH-specific plasma N-glycan signature, which was not only higher in AIH patients as compared to HC, but also as compared to other liver disease groups in the cohort. Only patients with viral or alcoholic hepatitis without cirrhosis featured high levels of A4GS as well, but in the clinic the differentiation between viral or alcoholic hepatitis and AIH is straightforward using conventional diagnostics. The observed effect was specific for tetraantennary glycans, and independent of sialic acid linkage. Of note, the sialylation per galactose of di-(A2GS) and triantennary glycans (A3GS) did not show the above-described trend, indicating it is not a global sialylation effect.

Sialylated tetraantennary glycans in human plasma largely originate from alpha-1-acid glycoprotein (AGP)²³. AGP is a major positive acute phase protein, that functions both as an immunomodulatory as well as transport protein, harbours 5 N-glycosylation sites and is characterized by remarkable glycosylation microheterogeneity²⁵⁻²⁷. Hitherto, reports on AGP glycosylation mainly highlighted alterations in branching and antennary fucosylation to be associated with (liver) diseases^{28,29}. The increased level of sialylation on tetraantennary glycans found in the plasma of AIH patients is a novel finding, which can either be an effect of altered regulation of sialylation or a proxy showing the upregulation of highly sialylated glycoproteins, such as AGP. Factors that regulate the levels of sialylation in human plasma are, amongst others, the abundance and activity of sialyltransferases in the glycoprotein producing cells, the availability of CMP-sialic acid and the removal of non-sialylated proteins from the circulation by the asialoglycoprotein receptor (ASGPR) in the liver. While investigating the expression levels of sialyltransferases in liver cells associated with AIH would be a fruitful direction for further research, a role of the ASGPR in the observed effects seems unlikely. AIH is associated with an upregulation of ASGPR-specific autoantibodies³⁰, which would have an opposite effect on the level of sialylation in the circulation, as the sialylated proteins will be limitedly removed.

In addition, the new marker has the potential to increase in specificity when the plasma/liver glycoproteins are identified that are responsible for the observed differences. An obvious glycoprotein candidate to further investigate is AGP and efforts to study levels and glycosylation of AGP in AIH patients are highly recommended. To consolidate our findings on the longitudinal intra-patient variation observed for A4GS and its potential to monitor disease activity over time, future studies should involve longitudinal samples. Despite the long-term follow-up in the current study, samples were intermittently distributed in time and showed a large heterogeneity in inflammation, cirrhosis, and treatment status as well as treatment type. This, in combination with limited sample numbers, may have caused an inherent bias when longitudinal changes in altering inflammation categories were compared.

Other differentiators between AIH patients and HC were TPNG- and IgG bisection. The degree of cirrhosis in liver diseases vastly confounded the bisection effect in our study and this trait appeared rather cirrhosis-specific than AIH-specific, as described in literature, and exploited in the clinically approved GlycoCirrhoTest^{7,8}. The bisection effect is likely (partly) derived from IgG, as supported by the IgG-specific data, but IgM and IgA, that are also known to carry diantennary glycans with bisecting N-acetylglucosamines²³ may contribute to this observation. The level of hybrid type glycans in plasma associated with the occurrence of cirrhosis and showed a trend towards increased levels with flaring AIH. However, for the evaluation of THy as marker for disease activity, replication is needed in a more uniform set of longitudinal follow-up samples, accounting for cirrhosis and the use of medication. Higher levels of hybrid-type structures are worth further investigation in the context of AIH, as hybrid-type glycans on vitronectin have been reported to be elevated in hepatocellular carcinoma patients^{31,32}. Conversely, plasma derived hybrid type glycans negatively associated with Crohn's disease and ulcerative colitis in a cross-sectional study⁹.

Increased plasma IgG levels are one of the hallmarks of AIH, which motivated the independent investigation of IgG-specific glycosylation. Part of the galactosylation effects observed in plasma (a decreased A2G, CG, A2FG in most liver diseases) were explained by IgG-specific changes²³. The undergalactosylation of IgG is a known marker of ongoing inflammation, which is well documented in a broad range of diseases such as RA^{33,34} and other autoimmune diseases³⁵⁻³⁷, IBD^{9,15}, colorectal cancer³⁸, infectious diseases^{39,40}, as well as upon aging^{17-19,41}. The underlying biological mechanism that might be responsible for the pro-inflammatory nature of undergalactosylated IgG is its potential ability to elicit complement activation via binding to the mannose-binding lectin¹⁴. However, this causal relationship was not proven and agalactosylated total plasma IgG can also be a mere effect of the inflammatory process. In addition, complement activation does not appear to play a major role in AIH⁴². There is a large body of evidence correlating IgG galactosylation with inflammatory markers and disease activity^{41,43,44}, and a lower IgG1 galactosylation correlated to disease activity in the current cross-sectional samples. However, no galactosylation effect was observed in the longitudinal sampling of patients changing their inflammatory status, which suggests that AIH patients in this cohort show too much heterogeneity in disease activity, cirrhosis severity and use of medication to use IgG galactosylation as marker for inflammation.

Our study focused on total plasma N-glycosylation and IgG glycosylation in an integrated fashion, which is unique in the context of high-throughput glycomics. Still, further studies are needed to confirm the source of protein from which the observed A4GS effect derives. The currently used glycoanalytical methodologies are not yet suitable for direct clinical application. As the clinically approved GlycoCirrhoTest unfortunately does not feature sialylation analysis^{7,8}, suitable alternatives should be developed to further implement glycosylation analysis in clinical practice. These may e.g. be based on (immune)affinity assays targeting sialylation, or exploit techniques that provide a higher glycoform resolution, such as capillary electrophoresis.

In conclusion, by characterizing total plasma N-glycosylation and IgG glycosylation, an AIH-specific glycosylation profile was found. High A4GS is unique for AIH, which offers possibilities for development of new diagnostic markers, potentially reducing the need for liver biopsy. Secondly, it can also offer a new perspective on pathophysiology of AIH. Glycosidic changes related to disease activity should be investigated further and might aid physicians with monitoring of disease activity in the future.

### REFERENCES

1 van Gerven, N. M., de Boer, Y. S., Mulder, C. J., van Nieuwkerk, C. M. & Bouma, G. Autoimmune hepatitis. World J Gastroenterol 22, 4651-4661, doi:10.3748/wjg.v22.i19.4651 (2016).
2 Alvarez, F. et al. International Autoimmune Hepatitis Group Report: review of criteria for diagnosis of autoimmune hepatitis. Journal of Hepatology 31, 929-938, doi:10.1016/s0168-8278(99)80297-9 (1999).
3 Hennes, E. M. et al. Simplified criteria for the diagnosis of autoimmune hepatitis. Hepatology 48, 169-176, doi:10.1002/hep.22322 (2008).
4 Lohse, A. W. et al. EASL Clinical Practise Guideline Autoimmune Hepatitis. J Hepatol 63, 971-1004, doi:2015; 63(4):971-1004 (2015).
Biewenga, M. et al. Aminotransferases during treatment predict long-term survival in patients with autoimmune hepatitis type 1: a landmark analysis. Clin Gastroenterol Hepatol, doi:10.1016/j.cgh.2021.05.024 (2021).
6 van Gerven, N. M. et al. Relapse is almost universal after withdrawal of immunosuppressive medication in patients with autoimmune hepatitis in remission. J Hepatol 58, 141-147, doi:10.1016/j.jhep.2012.09.009 (2013).
7 Callewaert, N. et al. Noninvasive diagnosis of liver cirrhosis using DNA sequencer-based total serum protein glycomics. Nat Med 10, 429-434, doi:10.1038/nm1006 (2004).
8 Verhelst, X. et al. A Glycomics-Based Test Predicts the Development of Hepatocellular Carcinoma in Cirrhosis. Clin Cancer Res 23, 2750-2758, doi:10.1158/1078-0432.CCR-16-1500 (2017).
9 Clerc, F. et al. Plasma N-Glycan Signatures Are Associated With Features of Inflammatory Bowel Diseases. Gastroenterology 155, 829-843, doi:10.1053/j.gastro.2018.05.030 (2018).
10 Dotz, V. et al. Plasma protein N-glycan signatures of type 2 diabetes. Biochimica et Biophysica Acta (BBA)—General Subjects 1862, 2613-2622, doi:https://doi.org/10.1016/j.bbagen.2018.08.005 (2018).
11 Verhelst, X. et al. Protein Glycosylation as a Diagnostic and Prognostic Marker of Chronic Inflammatory Gastrointestinal and Liver Diseases. Gastroenterology 158, 95-110, doi:10.1053/j.gastro.2019.08.060 (2020).
12 Blomme, B., Van Steenkiste, C., Callewaert, N. & Van Vlierberghe, H. Alteration of protein glycosylation in liver diseases. J Hepatol 50, 592-603, doi:10.1016/j.jhep.2008.12.010 (2009).
13 Subedi, G. P. & Barb, A. W. The Structural Role of Antibody N-Glycosylation in Receptor Interactions. Structure 23, 1573-1583, doi:10.1016/j.str.2015.06.015 (2015).
14 Malhotra, R. et al. Glycosylation changes of IgG associated with rheumatoid arthritis can activate complement via the mannose-binding protein. Nat Med 1, 237-243, doi:10.1038/nm0395-237 (1995).
15 Šimurina, M. et al. Glycosylation of Immunoglobulin G Associates With Clinical Features of Inflammatory Bowel Diseases. Gastroenterology 154, 1320-1333.e1310, doi:10.1053/j.gastro.2018.01.002 (2018).
16 Plomp, R. et al. Subclass-specific IgG glycosylation is associated with markers of inflammation and metabolic health. Sci Rep 7, 12325, doi:10.1038/s41598-017-12495-0 (2017).
17 de Haan, N., Reiding, K. R., Driessen, G., van der Burg, M. & Wuhrer, M. Changes in Healthy Human IgG Fc-Glycosylation after Birth and during Early Childhood. Journal of Proteome Research 15, 1853-1861, doi:10.1021/acs.jproteome.6b00038 (2016).
18 Knezevic, A. et al. Effects of aging, body mass index, plasma lipid profiles, and smoking on human plasma N-glycans. Glycobiology 20, 959-969, doi:10.1093/glycob/cwq051 (2010).
19 Vanhooren, V. et al. N-glycomic changes in serum proteins during human aging. Rejuvenation Res 10, 521-531a, doi:10.1089/rej.2007.0556 (2007).
20 Vreeker, G. C. M. et al. Automated Plasma Glycomics with Linkage-Specific Sialic Acid Esterification and Ultrahigh Resolution MS. Anal Chem 90, 11955-11961, doi:10.1021/acs.analchem.8b02391 (2018).
21 Falck, D., Jansen, B. C., de Haan, N. & Wuhrer, M. High-Throughput Analysis of IgG Fc Glycopeptides by LC-MS. Methods Mol Biol 1503, 31-47, doi:10.1007/978-1-4939-6493-2_4 (2017).
22 de Haan, N. et al. Differences in IgG Fc Glycosylation Are Associated with Outcome of Pediatric Meningococcal Sepsis. mBio 9, doi:10.1128/mBio.00546-18 (2018).
23 Clerc, F. et al. Human plasma protein N-glycosylation. Glycoconj J 33, 309-343, doi:10.1007/s10719-015-9626-2 (2016).
24 Wuhrer, M. et al. Glycosylation profiling of immunoglobulin G (IgG) subclasses from human serum. Proteomics 7, 4070-4081, doi:10.1002/pmic.200700289 (2007).
25 Takakura, D., Harazono, A., Hashii, N. & Kawasaki, N. Selective glycopeptide profiling by acetone enrichment and LC/MS. J Proteomics 101, 17-30, doi:10.1016/j.jprot.2014.02.005 (2014).
26 Imre, T. et al. Glycosylation site analysis of human alpha-1-acid glycoprotein (AGP) by capillary liquid chromatography-electrospray mass spectrometry. J Mass Spectrom 40, 1472-1483, doi:10.1002/jms.938 (2005).
27 Treuheit, M. J., Costello, C. E. & Halsall, H. B. Analysis of the five glycosylation sites of human alpha 1-acid glycoprotein. Biochem J 283 (Pt 1), 105-112, doi:10.1042/bj2830105 (1992).
28 Anderson, N. et al. A preliminary evaluation of the differences in the glycosylation of alpha-1-acid glycoprotein between individual liver diseases. Biomed Chromatogr 16, 365-372, doi:10.1002/bmc.167 (2002).
29 Ceciliani, F. & Pocacqua, V. The acute phase protein alpha1-acid glycoprotein: a model for altered glycosylation during diseases. Curr Protein Pept Sci 8, 91-108, doi:10.2174/138920307779941497 (2007).
30 Rigopoulou, E. I. et al. Asialoglycoprotein receptor (ASGPR) as target autoantigen in liver autoimmunity: lost and found. Autoimmun Rev 12, 260-269, doi:10.1016/j.autrev.2012.04.005 (2012).
31 Lee, H. J. et al. Abundance-ratio-based semiquantitative analysis of site-specific N-linked glycopeptides present in the plasma of hepatocellular carcinoma patients. J Proteome Res 13, 2328-2338, doi:10.1021/pr4011519 (2014).
32 Hwang, H. et al. In-depth analysis of site-specific N-glycosylation in vitronectin from human plasma by tandem mass spectrometry with immunoprecipitation. Anal Bioanal Chem 406, 7999-8011, doi:10.1007/s00216-014-8226-5 (2014).
33 Parekh, R. B. et al. Association of rheumatoid arthritis and primary osteoarthritis with changes in the glycosylation pattern of total serum IgG. Nature 316, 452-457, doi:10.1038/316452a0 (1985).
34 Ercan, A. et al. Aberrant IgG galactosylation precedes disease onset, correlates with disease activity, and is prevalent in autoantibodies in rheumatoid arthritis. Arthritis Rheum 62, 2239-2248, doi:10.1002/art.27533 (2010).
35 Sonneveld, M. E. et al. Patients with IgG1-anti-red blood cell autoantibodies show aberrant Fc-glycosylation. Sci Rep 7, 8187, doi:10.1038/s41598-017-08654-y (2017).
36 Vuckovic, F. et al. Association of systemic lupus erythematosus with decreased immunosuppressive potential of the IgG glycome. Arthritis Rheumatol 67, 2978-2989, doi:10.1002/art.39273 (2015).
37 Selman, M. H. et al. IgG fc N-glycosylation changes in Lambert-Eaton myasthenic syndrome and myasthenia gravis. J Proteome Res 10, 143-152, doi:10.1021/pr1004373 (2011).
38 Vuckovic, F. et al. IgG Glycome in Colorectal Cancer. Clin Cancer Res 22, 3078-3086, doi:10.1158/1078-0432.CCR-15-1867 (2016).
39 Ackerman, M. E. et al. Natural variation in Fc glycosylation of HIV-specific antibodies impacts antiviral activity. J Clin Invest 123, 2183-2192, doi:10.1172/JC165708 (2013).
40 Parekh, R. et al. A comparative analysis of disease-associated changes in the galactosylation of serum IgG. Journal of Autoimmunity 2, 101-114, doi:10.1016/0896-8411(89)90148-0 (1989).
41 Gudelj, I., Lauc, G. & Pezer, M. Immunoglobulin G glycosylation in aging and diseases. Cell Immunol 333, 65-79, doi:10.1016/j.cellimm.2018.07.009 (2018).
42 Biewenga, M. et al. The role of complement activation in autoimmune liver disease. Autoimmun Rev 19, 102534, doi:10.1016/j.autrev.2020.102534 (2020).
43 Gornik, O., Pavic, T. & Lauc, G. Alternative glycosylation modulates function of IgG and other proteins—implications on evolution and disease. Biochim Biophys Acta 1820, 1318-1326, doi:10.1016/j.bbagen.2011.12.004 (2012).
44 Cobb, B. A. The history of IgG glycosylation and where we are now. Glycobiology 30, 202-213, doi:10.1093/glycob/cwz065 (2020).
45 Bladergroen, M. R. et al. Automation of High-Throughput Mass Spectrometry-Based Plasma N-Glycome Analysis with Linkage-Specific Sialic Acid Esterification. Journal of Proteome Research 14, 4080-4086, doi:10.1021/acs.jproteome.5b00538 (2015).
46 Singh, S. S. et al. Metformin and statin use associate with plasma protein N-glycosylation in people with type 2 diabetes. BMJ Open Diabetes Res Care 8, doi:10.1136/bmjdrc-2020-001230 (2020).
47 Jansen, B. C. et al. MassyTools: A High-Throughput Targeted Data Processing Tool for Relative Quantitation and Quality Control Developed for Glycomic and Glycoproteomic MALDI-MS. J Proteome Res 14, 5088-5098, doi:10.1021/acs.jproteome.5b00658 (2015).
48 Jansen, B. C. et al. LaCyTools: A Targeted Liquid Chromatography-Mass Spectrometry Data Processing Package for Relative Quantitation of Glycopeptides. J Proteome Res 15, 2198-2210, doi:10.1021/acs.jproteome.6b00171 (2016)
49 Klein, A. Human total serum N-glycome. Adv Clin Chem 46, 51-85, doi:doi.org/10.1016/S0065-2423(08)00402-2 (2008).
50 Pucic, M. et al. High throughput isolation and glycosylation analysis of IgG-variability and heritability of the IgG glycome in three isolated human populations. Mol Cell Proteomics 10, M111 010090, doi:10.1074/mcp.M111.010090 (2011).

What is claimed is:

1. A method for diagnosing autoimmune hepatis (AIH) in a subject, or predicting the risk of AIH developing in a subject, the method comprising:
a) determining the level of sialylation per galactose on tetraantennary glycans (A4GS) in a sample from the subject; and
b) comparing the level of A4GS in the sample to a reference level, wherein an increase in the level of A4GS in the sample as compared to the reference level is indicative of the subject having AIH, or being at risk of developing AIH.

2. A method for diagnosing AIH in a subject, or predicting the risk of the AIH developing in a subject, and treating AIH in a subject, the method comprising:
a) determining the level of A4GS in a sample from the subject;
b) comparing the level of A4GS in the sample to a reference level;
c) identifying the subject as having AIH if the levels of A4GS in the sample are increased compared to the reference level; and
d) administering to the subject that has been identified as having AIH or being at risk of having AIH a treatment for AIH.

3. The method of claim 2, wherein the sample is a biological fluid sample.

4. The method of claim 3, wherein the sample is selected from the group consisting of blood, urine and saliva, optionally wherein the blood sample is plasma or whole blood.

5. The method of claim 4, wherein the sample is a plasma sample and has been subjected to N-glycans release from plasma proteins and linkage-specific chemical sialic acid derivatization.

6. The method of claim 2, wherein the subject is a mammal, optionally wherein the subject is a human, a monkey, a rat, a mouse, a mink, a rabbit, a guinea pig, a pig, a dog, a cat, a goat, a sheep, a horse or a cow.

7. The method of claim 2, wherein the subject does not have, or is not believed to have, viral or alcoholic hepatitis.

8. The method of claim 2, wherein the subject is symptomatic or asymptomatic.

9. The method of claim 8, wherein the symptomatic subject has a symptom selected from the group consisting of fatigue, jaundice, abdominal pain, joint pain and/or swelling, mild flu-like symptoms, itching, large abdomen due to enlarged liver and/or spleen, and spiderlike blood vessels in the skin.

10. The method of claim 2, wherein the level A4GS is determined by mass spectrometry, high-performance liquid chromatography, capillary (gel) electrophoresis with laser induced fluoresces detection, hydrophilic interaction liquid chromatography (for example with fluorescence or UV detection), or an ELISA based assay (for example an ELISA based assay using glycan binding proteins such as lectins or antibodies).

11. The method of claim 2, wherein the method further comprises determining the level of aspartate transaminase (AST), alanine transaminase (ALT) and/or IgG.

12. A method of treating AIH in a subject comprising: administering an AIH treatment to a subject that has been diagnosed or determined to be at risk of AIH, or has relapsed, by the method of claim 1.

13. The method of claim 12, wherein the treatment is a prophylactic treatment or disease modifying treatment.

14. The method of claim 12, wherein the treatment is an immunosuppressant, optionally the immunosuppressant is prednisone, prednisolone, budesonide, azathioprine, mycophenolate mofetil, 6-thioguanine, 6-mercaptopurine, rituximab and/or tacrolimus.

15. A kit for use in the method of claim 2, the kit comprising a detectably labelled agent that specifically binds to A4GS.

16. The kit of claim 15 comprising a detectably labelled agent that specifically binds to aspartate AST, ALT and/or IgG.

17. Use of A4GS as a biomarker for AIH.
